(19) United States
(12) Patent Application Publication
(10) Pub. No.: US 2026/0191412 A1
(43) Pub. Date: Jul. 9, 2026

(54) PORTABLE MAGNETIC RESONANCE IMAGING SYSTEM

(71) Applicant: Marsha Dillabough, Milton, FL (US)

(72) Inventor: Marsha Dillabough, Milton, FL (US)

(21) Appl. No.: 19/015,268

(22) Filed: Jan. 9, 2025

(51) Int. Cl. A61B 5/00; A61B 5/055; G01R 33/54

(52) U.S. Cl. CPC A61B 5/004; A61B 5/055; G01R 33/546

(57) ABSTRACT

A portable magnetic resonance imaging system includes a handheld scanner, a limb scanner, and a data storage device. The handheld scanner includes a scan head and a handle attached to the scan head. The scan head is designed to be positioned adjacent a patient for magnetic resonance imaging. A first data cable is coupled to the handle of the handheld scanner. The limb scanner includes a housing that is open-ended and designed to receive a limb of a patient for magnetic resonance imaging. A second data cable is coupled to the limb scanner. The data storage device is connectable to the first data cable and the second data cable.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to magnetic resonance imaging (MRI) devices and more particularly pertains to a new portable magnetic resonance imaging system for providing portable MRI procedures on specific parts of a body of a patient in essentially any location, whether inside a medical facility or outside. The portable magnetic resonance imaging system includes a handheld scanner for targeted MRI procedures and a limb scanner in the form of a portable tube for MRI procedures on a limb of a patient.

### (2) Description of Related Art Including Information Disclosed Under 37 CFR 1.97 and 1.98

The prior art relates to magnetic resonance imaging devices, which include handheld wands for use with tables in hospitals or medical offices as well as portable handheld wands for use in exterior locations. The prior art, as best understood, does not disclose a portable magnetic resonance imaging system that includes a handheld scanner, a limb scanner, and a data storage device. The handheld scanner includes a scan head and a handle attached to the scan head. The scan head is designed to be positioned adjacent a patient for magnetic resonance imaging. A first data cable is coupled to the handle of the handheld scanner. The limb scanner includes a housing that is open-ended and designed to receive a limb of a patient for magnetic resonance imaging. A second data cable is coupled to the limb scanner. The data storage device is connectable to the first data cable and the second data cable.

### (h) BRIEF SUMMARY OF THE INVENTION

An embodiment of the disclosure meets the needs presented above in a portable magnetic resonance imaging system generally comprising a portable magnetic resonance imaging system includes a handheld scanner, a limb scanner, and a data storage device. The handheld scanner includes a scan head and a handle attached to the scan head. The scan head is designed to be positioned adjacent a patient for magnetic resonance imaging. A first data cable is coupled to the handle of the handheld scanner. The limb scanner includes a housing that is open-ended and designed to receive a limb of a patient for magnetic resonance imaging. A second data cable is coupled to the limb scanner. The data storage device is connectable to the first data cable and the second data cable.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a portable magnetic resonance imaging system according to an embodiment of the disclosure.

FIG. 2 is a front view of an embodiment of the disclosure.

FIG. 3 is a side view of an embodiment of the disclosure.

FIG. 4 is a front view of an embodiment of the disclosure.

FIG. 5 is a perspective view of an embodiment of the disclosure in use.

FIG. 6 is a perspective view of an embodiment of the disclosure in use.

FIG. 7 is a front view of an embodiment of the disclosure in use.

### (j) DETAILED DESCRIPTION OF THE INVENTION

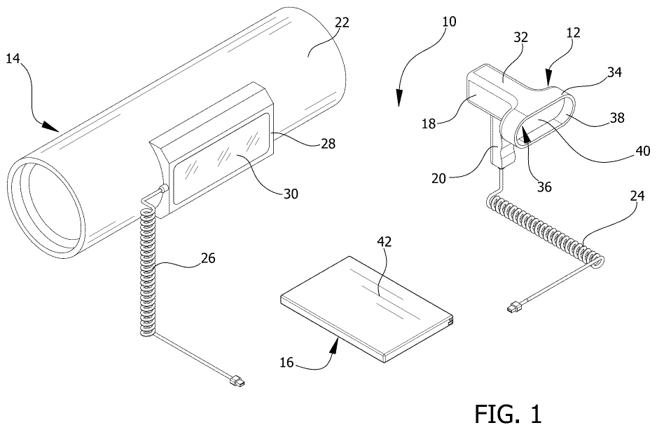

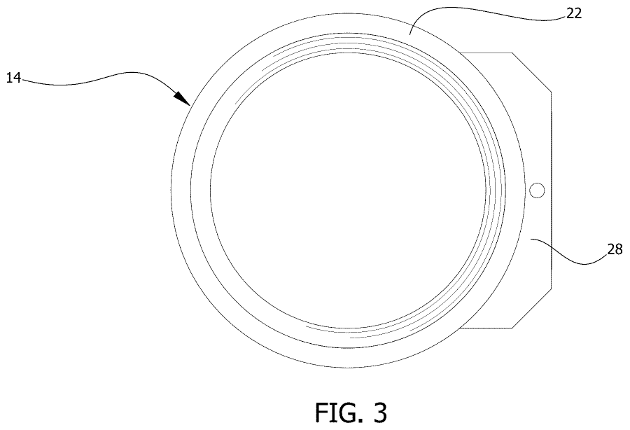

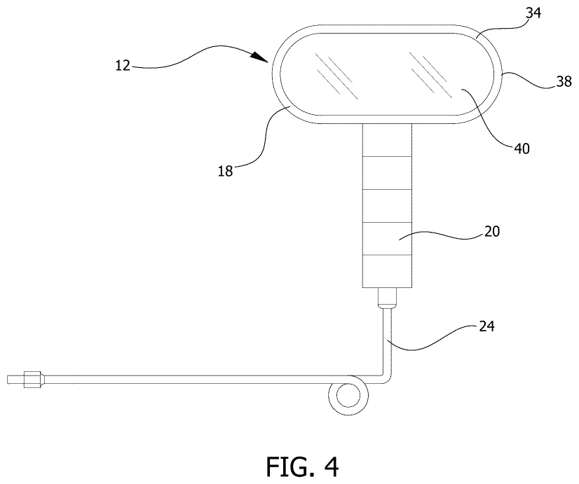

With reference now to the drawings, and in particular to FIGS. 1 through 7 thereof, a new portable magnetic resonance imaging system embodying the principles and concepts of an embodiment of the disclosure and generally designated by the reference numeral 10 will be described.

As best illustrated in FIGS. 1 through 7, the portable magnetic resonance imaging system 10 generally comprises a handheld scanner 12, a limb scanner 14, and a data storage device 16. The handheld scanner 12 includes a scan head 18 and a handle 20 attached to the scan head 18. The scan head 18 is designed to be positioned adjacent a patient 70 for magnetic resonance imaging. A first data cable 24 is coupled to the handle 20 of the handheld scanner 12. The limb scanner 14 includes a housing 22 that is open-ended and designed to receive a limb of the patient 70 for magnetic resonance imaging. A second data cable 26 is coupled to the limb scanner 14. The data storage device 16 is connectable to the first data cable 24 and the second data cable 26.

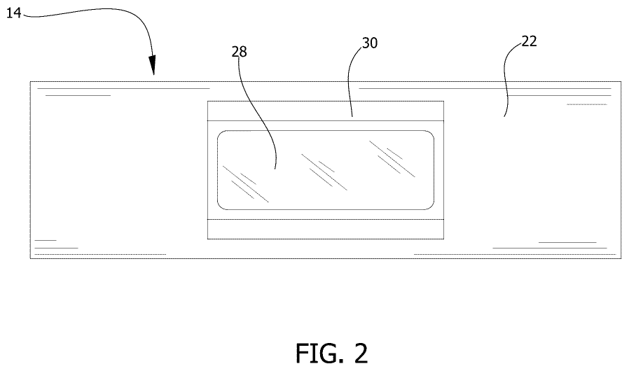

In the exemplary embodiment in FIG. 2, the limb scanner 14 includes a control interface 28 attached to the housing 22. The second data cable 26 is coupled to the control interface 28, as shown in FIG. 1. The control interface 28 includes a touch screen 30 designed for user input. The user can therefore input such information as operating commands or scanning parameters via the touch screen 30 for control of the limb scanner 14. The control interface 28 could also includes manual buttons or switches.

The scan head 18 includes a body portion 32 and a head portion 34. The head portion 34 is wider than the body portion 32 and positioned perpendicular to the body portion 32. The head portion 34 includes an opening 36, a lip 38 around the opening 36, and a wall 40 recessed from an edge of the lip 38. The body portion 32 is elongated and the handle 20 projects from the body portion 32 perpendicularly to a longitudinal dimension of the body portion 32. The handheld scanner 12 therefore has a pistol-style design much like a barcode scanner or similar, which enables a more ergonomic grip and better control over the direction of the scanning procedure.

In the exemplary embodiment in FIG. 1, the data storage device 16 is a portable hard drive 42. The portable hard drive 42 can include multiple data ports, as is well known in the computer arts, for connection to the first data cable 24 and the second data cable 26, either individually or simultaneously.

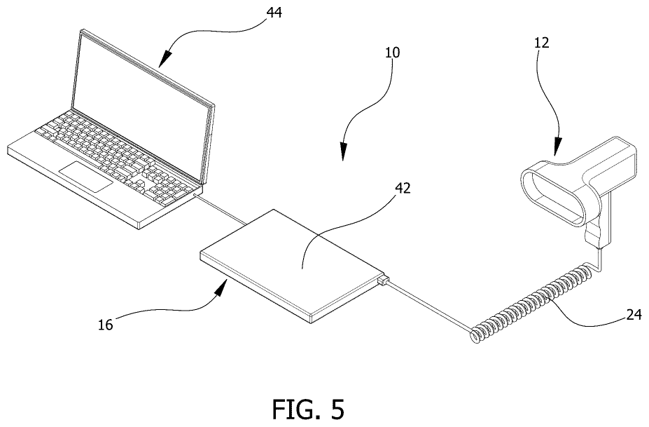

In the exemplary embodiment in FIG. 5, the portable magnetic resonance imaging system 10 further includes a laptop computer 44. In one possible embodiment, the laptop computer 44 could be replaced with a tablet or similar portable computing device. In one possible embodiment, the data storage device 16 can be a hard drive on the laptop computer 44. In the exemplary embodiment in FIG. 5, the data storage device 16 is the portable hard drive 42, which is connected to the laptop computer 44. In one possible embodiment, the laptop computer 44 is designed to automatically operate the handheld scanner 12 and the limb scanner 14 and display magnetic resonance images on a screen of the laptop computer 44.

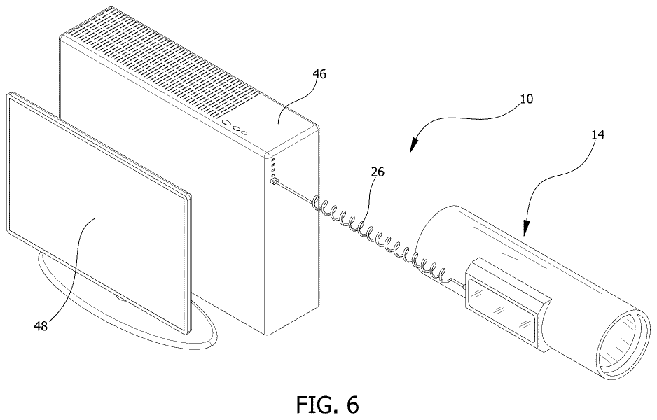

In the exemplary embodiment in FIG. 6, the portable magnetic resonance imaging system 10 is being used with a desktop computer 46. In this example, the limb scanner 14 is directly connected to the desktop computer 46, which is connected to a monitor 48. The monitor 48 can display MRI images.

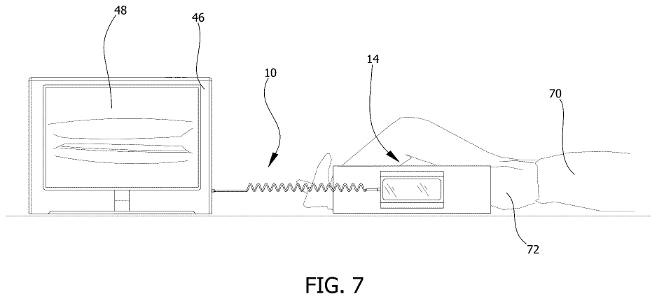

The portable magnetic resonance imaging system 10 can be used either in a medical facility or outside a medical facility. The handheld scanner 12 is used for scanning a desired part of a body of the patient 70. The limb scanner 14 is used for scanning a limb, such as an arm or a leg. FIG. 7 shows the limb scanner 14 being used to scan a leg 72, which has been inserted into the housing 22. If using just the portable hard drive 42, the portable hard drive 42 can store MRI images for reading later on a computer, such as the laptop computer 44 or the desktop computer 46. If using the laptop computer 44, the MRI images can be stored or viewed in real time. The same can be done with the desktop computer 46, as shown in FIG. 7, wherein the MRI image is viewable on the monitor 48.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A portable magnetic resonance imaging system comprising:
a handheld scanner, said handheld scanner including a scan head and a handle attached to said scan head, said scan head being configured to be positioned adjacent a patient for magnetic resonance imaging;
a first data cable being coupled to said handle of said handheld scanner;
a limb scanner, said limb scanner including a housing being open-ended and configured to receive a limb of a patient for magnetic resonance imaging;
a second data cable being coupled to said limb scanner; and
a data storage device being connectable to said first data cable and said second data cable.

2. The portable magnetic resonance imaging system of claim 1, wherein:
said limb scanner includes a control interface attached to said housing; and
said second data cable is coupled to said control interface.

3. The portable magnetic resonance imaging system of claim 2, wherein said control interface includes a touch screen configured for user input.

4. The portable magnetic resonance imaging system of claim 1, wherein:
said scan head includes a body portion and a head portion; and
said head portion is wider than said body portion and positioned perpendicular to said body portion.

5. The portable magnetic resonance imaging system of claim 4, wherein said head portion includes an opening, a lip around said opening, and a wall recessed from an edge of said lip.

6. The portable magnetic resonance imaging system of claim 4, wherein said body portion is elongated and said handle projects from said body portion perpendicularly to a longitudinal dimension of said body portion.

7. The portable magnetic resonance imaging system of claim 1, wherein said data storage device is a portable hard drive.

8. The portable magnetic resonance imaging system of claim 1, further including a laptop computer, wherein said data storage device is a hard drive on said laptop computer.

9. The portable magnetic resonance imaging system of claim 8, wherein said laptop computer is configured to automatically operate said handheld scanner and said limb scanner and display magnetic resonance images on a screen of said laptop computer.

10. The portable magnetic resonance imaging system of claim 3, wherein:
said scan head includes a body portion and a head portion; and
said head portion is wider than said body portion and positioned perpendicular to said body portion.

11. The portable magnetic resonance imaging system of claim 10, wherein said head portion includes an opening, a lip around said opening, and a wall recessed from an edge of said lip.

12. The portable magnetic resonance imaging system of claim 11, wherein said body portion is elongated and said handle projects from said body portion perpendicularly to a longitudinal dimension of said body portion.

13. The portable magnetic resonance imaging system of claim 12, wherein said data storage device is a portable hard drive.

14. The portable magnetic resonance imaging system of claim 12, further including a laptop computer, wherein said data storage device is a hard drive on said laptop computer.

15. The portable magnetic resonance imaging system of claim 14, wherein said laptop computer is configured to automatically operate said handheld scanner and said limb scanner and display magnetic resonance images on a screen of said laptop computer.
